(19) United States
(12) Patent Application Publication
(10) Pub. No.: US 2024/0271078 A1
(43) Pub. Date: Aug. 15, 2024

(54) ASSESSING PACKED CELL VOLUME FOR CELL CULTURES

(71) Applicant: AMGEN INC., Thousand Oaks, CA (US)

(72) Inventors: Jeremy S. Conner, Newbury Park, CA (US); Sarah Whetstone, Thousand Oaks, CA (US); Ketan Kumar, Cambridge, MA (US)

(21) Appl. No.: 18/567,750

(22) PCT Filed: Jun. 6, 2022
(86) PCT No.: PCT/US22/32308

§ 371 Date: Dec. 6, 2023

Related U.S. Application Data
(60) Provisional application No. 63/208,824, filed on Jun. 9, 2021.

(51) Int. Cl. C12M 1/36; C12M 1/34

(52) U.S. Cl. CPC C12M 41/48; C12M 41/36; C12M 41/46

(57) ABSTRACT

A method of cell culture assessment (e.g., prior to a drug substance harvesting process) includes obtaining a plurality of parameters associated with a cell culture, and inferring or predicting a value or classification indicative of packed cell volume. Inferring or predicting the packed cell volume includes applying the plurality of parameters as inputs to a non-linear machine learning model. The method also includes generating an output indicative of the inferred or predicted value or classification.

### FIELD OF THE DISCLOSURE

The present application relates generally to cell cultures (e.g., in a bioreactor), and more specifically to the explicit or implicit determination of packed cell volume.

### BACKGROUND

In the manufacture of certain biopharmaceutical products (e.g., biotherapeutic proteins), bioreactors are used to culture cells prior to harvesting a desired drug substance. Daily sampling from such bioreactors is typically required in process development laboratories, in order to ensure cell health and continuity between runs. This sampling process typically involves manually removing a sample of cell culture from the bioreactor, and then utilizing multiple analytical instruments, a centrifuge, and a manual process to visually measure the packed cell volume. A conventional implementation of this sampling process is shown as sampling process 200 in FIG. 2. In the process 200, immediately after a sample is removed from the bioreactor, the sample is taken to a blood gas analyzer (BGA) to measure pH, pO2, and pCO2. The cell-containing sample is then pipetted into an analyzer for cell measurements, such as total cell count (TCC), viable cell density (VCD), and cell diameter. An additional measurement to determine packed cell volume (PCV) is typically required on specific days of the process. However, the process for measuring packed cell volume is more labor-intensive than any other single measurement required using the sample aliquots. Moreover, unlike the measurements noted above, the packed cell volume measurement does not lend itself to automated sampling, due to the required interaction with a centrifuge (e.g., specially designed centrifuge spin tubes) and the nature of the visual interpretation of results. Other analytical measurements, such as the titer measurements shown in FIG. 2, can also require the use of a centrifuge.

On days that packed cell volume is measured, the sample is typically divided into two separate tubes to be centrifuged. The supernatant of the first tube is removed and taken to devices for analytical osmolality and metabolite measurements. The second tube, which is dedicated to packed cell volume measurement, is spun down in a centrifuge. An individual then estimates packed cell volume by visually approximating the percentage of solid cells compacted at the bottom of the spun centrifuge tube in relation to the total volume of cells and liquid in the tube. This daily sampling/analysis process can be very time consuming (e.g., roughly three hours for one person assessing samples from a row of eight bioreactors). Furthermore, the subjective visual estimation process can result in relatively low packed cell volume accuracy, substantial variance between the visual estimations made by different individuals, and possibly substantial variance between the visual estimations made by a single individual at different times.

### BRIEF SUMMARY

Systems and methods described herein use a non-linear machine learning model (e.g., a neural network) to infer or predict a value or classification indicative of packed cell volume. For example, the model may infer from measured cell culture characteristics (e.g., experiment day, viable cell density, and viability) that the cell culture has a specific packed cell volume (e.g., expressed as a percentage), or may predict that a hypothetical cell culture with those characteristics would have a specific packed cell volume. Alternatively, the packed cell volume may be only implicitly determined or estimated. For example, the model may predict from measured cell culture characteristics that the cell culture would likely clog the filter during the harvesting process due to high packed cell volume, or that the cell culture would require modification to the timing, feed rate, and/or other parameters associated with a device (e.g., centrifuge or pump) used during the harvesting process. In some embodiments, the inferred or predicted value or classification is used to generate control data for one or more devices used in the harvesting process.

The techniques disclosed herein may obviate or lessen the need for manual assessments of packed cell volume, and may obviate or lessen the need for cell culture sampling (automated or otherwise) for purposes of packed cell volume assessments. Moreover, the disclosed techniques may more accurately estimate packed cell volumes, more reliably predict downstream issues (e.g., whether problems are likely to occur during harvesting, or whether/how centrifuge parameters should be modified to avoid such problems, etc.), and/or control downstream processes in a manner that achieves better performance (e.g., higher harvest yield).

### BRIEF DESCRIPTION OF THE DRAWINGS

The skilled artisan will understand that the figures described herein are included for purposes of illustration and are not limiting on the present disclosure. The drawings are not necessarily to scale, emphasis instead being placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 is a block diagram of an example system that may be used to explicitly or implicitly assess packed cell volume of a cell culture.

FIG. 2 depicts a conventional implementation of a cell culture sampling process prior to drug substance harvesting.

FIG. 3 is an example user interface that may be used to enter cell culture parameters and to view inferred or predicted packed cell volumes.

FIG. 4A is an example plot comparing actual and inferred packed cell volumes for a linear regression model.

FIG. 4B is an example plot comparing actual and inferred packed cell volumes for a random forest model.

FIG. 5 is a block diagram of an example system that may be used to control one or more devices for a cell culture harvesting process.

FIG. 6 is a flow diagram of an example method of cell culture assessment that may occur, for example, prior to a drug substance harvesting process.

### DETAILED DESCRIPTION

The various concepts introduced above and discussed in greater detail below may be implemented in any of numerous ways, and the described concepts are not limited to any particular manner of implementation. Examples of implementations are provided for illustrative purposes.

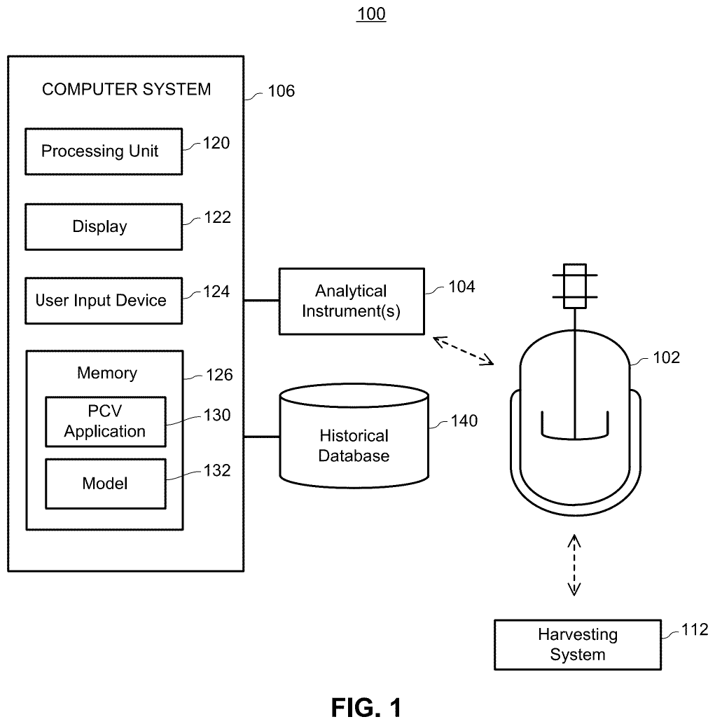

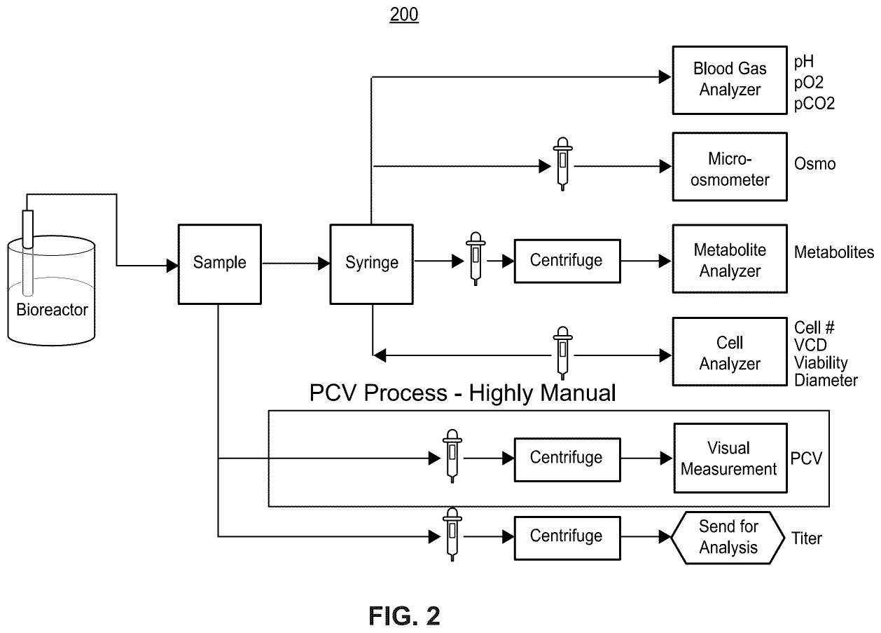

FIG. 1 is a simplified block diagram of an example system 100 that may be used to analyze (explicitly or implicitly) the packed cell volume of a cell culture. The cell culture may be a real-world/actual culture or a hypothetical culture, and the analysis may be for any of various purposes, depending on the embodiment and/or scenario. For example, the system 100 may be used to assess a bioreactor cell culture to determine whether the packed cell volume is too high for a particular filtering step of a harvesting process. As another example, for research and development purposes, the system 100 may be used to assess the effects of certain cell culture attributes on packed cell volume, regardless of whether any cell culture with those attributes presently exists. For ease of explanation, however, the system 100 is shown and described primarily with respect to an embodiment in which the system 100 assesses a real-world cell culture in a bioreactor 102, prior to a harvesting process.

In addition to the bioreactor 102, the system 100 includes one or more analytical instruments 104, a computer system 106, and a harvesting system 112. The bioreactor 102 may be any suitable vessel, device, or system that supports a cell culture, which may include living cells and/or substances derived therefrom within a media. The bioreactor 102 may contain recombinant proteins that are being expressed by the cell culture, e.g., such as for research purposes, clinical use, commercial sale, or other distribution. Depending on the biopharmaceutical process taking place, the media may include a particular fluid (e.g., a “broth”) and specific nutrients, and may have a target pH level or range, a target temperature or temperature range, and so on.

The analytical instrument(s) 104, which may or may not be communicatively coupled to the computer system 106 depending on the embodiment, can include any in-line, at-line, and/or off-line instrument (or instruments) configured to measure one or more attributes of the cell culture within the bioreactor 102. For example, the analytical instrument(s) 104 may measure viable cell density (VCD), viability, total cell count (TCC), cell diameter, and/or osmolality. Additionally or alternatively, the analytical instrument(s) 104 may measure one or more media component concentrations, such as metabolite levels (e.g., glucose, lactate, sodium, potassium, glutamine, ammonium, etc.), and/or one or more other cell culture attributes associated with the contents of the bioreactor 102 (e.g., temperature, pH, etc.). While in some embodiments the analytical instrument(s) 104 may use destructive analysis techniques, in other embodiments one, some, or all of the analytical instrument(s) 104 use non-destructive analysis (e.g., “soft sensing”) techniques.

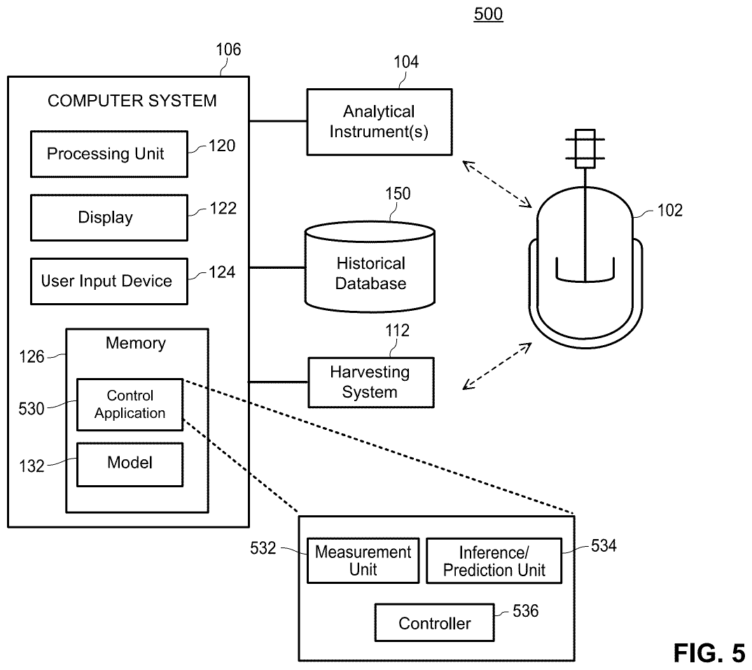

The harvesting system 112 includes one or more devices that are used during the harvest process, e.g., to extract a desired drug substance from the cell culture of the bioreactor 102. As used herein, the term “device” may refer to a free-standing device, a component integrated within another device or system, or any combination thereof. For example, the harvesting system 112 may include one or more filters, a pump, a centrifuge, and/or other suitable devices, any of which may be separate from or integrated within the bioreactor 102. In some embodiments, and as discussed in further detail below with reference to FIG. 5, one or more devices of the harvesting system 112 may be communicatively coupled to the computer system 106 for control purposes.

The computer system 106 may be a server, a desktop computer, a laptop computer, a tablet device, or any other suitable type of computing device or devices. In the example embodiment shown in FIG. 1, the computer system 106 includes a processing unit 120, a display device 122, a user input device 124, and a memory 126. In some embodiments, the computer system 106 includes two or more computers that are either co-located or remote from each other. For example, the computer system 106 may include both a server that hosts a web service, and a client device that accesses that web service. In distributed embodiments, the operations described herein relating to the processing unit 120 and/or the memory 126 may be divided among multiple processing units and/or memories, respectively, and/or the display device 122 and/or user input device 124 may reside at a computing device (e.g., client or terminal) that is separate from (e.g., remote from) at least a portion of the processing unit 120 and/or memory 126.

The processing unit 120 includes one or more processors, each of which may be a programmable microprocessor that executes software instructions stored in the memory 126 to execute some or all of the functions of the computer system 106 as described herein. Alternatively, some of the processors in the processing unit 120 may be other types of processors (e.g., application-specific integrated circuits (ASICs), field-programmable gate arrays (FPGAs), etc.), and some of the functionality of the computer system 106 as described herein may instead be implemented, in part or in whole, by such hardware. The memory 126 may include one or more physical memory devices or units containing volatile and/or non-volatile memory. Any suitable memory type or types may be used, such as read-only memory (ROM), solid-state drives (SSDs), hard disk drives (HDDs), and so on.

The display device 122 may use any suitable display technology (e.g., LED, OLED, LCD, etc.) to present information to a user, and the user input device 124 may be a keyboard or other suitable input device. In some embodiments, the display device 122 and the user input device 124 are integrated within a single device (e.g., a touchscreen display). Generally, the display device 122 and the user input device 124 mayjointly enable a user to interact with user interfaces (e.g., graphical user interfaces) provided by the computer system 106, e.g., to obtain an estimate of the packed cell volume of the cell culture process occurring within the bioreactor 102 as discussed in further detail below. In some embodiments, however, the computer system 106 does not include the display device 122 and/or the user input device 124.

The memory 126 stores the instructions of one or more software applications, including a packed cell volume (PCV) application 130, as well as data used by and generated by the software application(s). It is understood that the PCV application 130 may be a single software application executed by a single computing device, or may have various units or modules distributed among multiple software applications and/or executed by multiple computing devices. The PCV application 130, when executed by the processing unit 120, is generally configured to infer or predict packed cell volumes, either explicitly or implicitly, based on parameters associated with real-world and/or hypothetical cell cultures. To this end, the PCV application 130 implements a model 132, which may also be stored in the memory 126. The model 132 is a non-linear, machine learning model (e.g., a neural network, a random forest model, or an XGBoost model), which was trained by the computer system 106 (or by another suitable computing device or system) using data in a historical database 140.

The historical database 140 may be stored in the memory unit 126, and/or in one or more other persistent memories that are local or remote from each other (e.g., in a memory coupled to a remote library server, etc.). Generally, the historical database 140 may include numeric and/or categorical parameters associated with real-world cell cultures, such as experiment day (i.e., the count of days since a cell culture was introduced to a bioreactor, starting at Day 0 or Day 1), viable cell density (VCD), viability, total cell count (TCC), cell diameter, osmolality, molecule identifier (e.g., a particular antibody identifier), facility identifier (e.g., an identifier of the facility in which the cell culture was maintained), and/or other parameters (e.g., temperature, pH, particular metabolite levels, etc.). In some embodiments, the historical database 140 stores parameters corresponding to multiple different days of a single experiment (e.g., nine parameters corresponding to VCD at each of Days 0 through 8). Generally, the stored parameters can include any combination of known values (e.g., experiment day, molecule type), direct measurements, and/or indirect (e.g., soft-sensed) measurements and/or derived values, so long as at least some of the stored parameters are correlated in some way with (i.e., have some inferential or predictive strength with respect to) the packed cell volume of a cell culture.

In some embodiments, for each set of parameters associated with a particular experiment/cell culture, the historical database 140 also includes a label representing the packed cell volume corresponding to that set of parameters. The label may be a measured value, or a manually-estimated value (e.g., a packed cell volume estimate made by a skilled individual, or an average of such estimates from multiple individuals), for example. In other embodiments, the labels are not packed cell volume values, and instead are values or classifications that are indicative of packed cell volumes. For example, the labels may be classifications such as “adequate filter performance” or “inadequate filter performance” for a harvesting filter that requires a sufficiently low packed cell volume to avoid clogging. Labels of this sort may be set based on actual filter performance during harvesting (e.g., whether or not clogging actually occurred), or based on measured or estimated packed cell volumes and their known correspondences (e.g., labeling filter performance as “inadequate” in any case where the packed cell volume exceeded a threshold known to correspond to substantial filter clogging). As another example, the labels may be classifiers such as “modification required” or “no modification required,” to indicate whether modification of harvesting centrifuge parameters (e.g., timing parameters) was required as a result of excessive packed cell volume. As still another example, the labels may be specific centrifuge (or other device) parameters that were successfully used during harvesting, so long as those parameters are indicative of packed cell volume (e.g., with higher filter pressures corresponding to higher packed cell volumes, etc.).

The computer system 106, or another device or system, trains the model 132 using the cell culture parameters in the historical database 140 and the corresponding labels. For each set of parameters, for example, the computer system 106 may use the untrained or partially trained model 132 to infer or predict a value or classification, compare the value or classification to the corresponding label, and then modify the model 132 only if the label does not match the inferred or predicted value or classification (e.g., by modifying weights associated with the outputs of particular neural network nodes). In embodiments where a different computing device or system trains the model 132, the computer system 106 may obtain the model 132 via a wired or wireless communication network (e.g., via an Internet or intranet download), or via any other suitable means (e.g., by copying the model 132 from a portable storage medium). In some embodiments, the computer system 106 updates/refines the trained model 132 during operation, by using real-world results (e.g., additional packed cell volume estimates, or indications of whether filter clogging occurred, etc.) as labels for additional sets of model input data.

It is understood that other configurations and/or components may be used instead of those shown in FIG. 1. For example, a different computing device or system (not shown in FIG. 1) may transmit measurements provided by the analytical instrument(s) 104 to the computer system 106 via a network, one or more additional computing systems and one or more networks may act as intermediaries between the computer system 106 and the historical database 140, and so on.

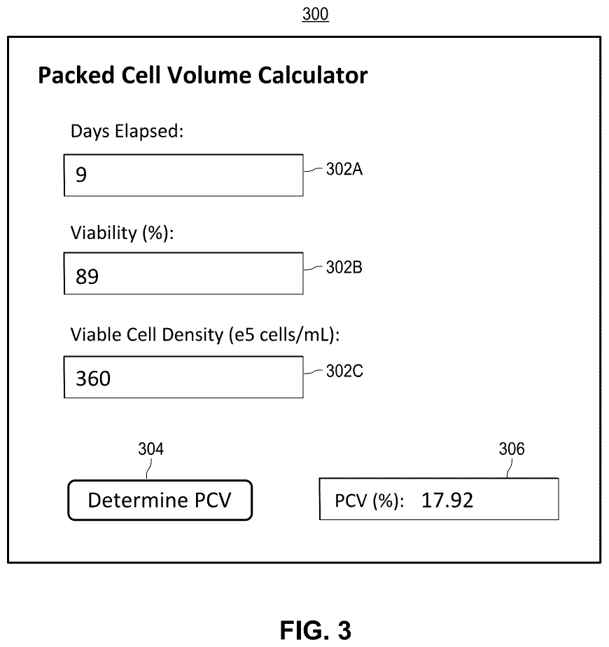

In operation, after the model 132 is trained, the PCV application 130 obtains parameters associated with a real-world or hypothetical cell culture. In some embodiments, the PCV application 130 obtains some or all of these parameters via a user interface generated by the PCV application 130. One example of such a user interface is the user interface 300 of FIG. 3, which may be generated by the processing unit 120 (e.g., when executing the instructions of the PCV application 130 stored in the memory 126) for presentation on the display 122. In some embodiments, the user interface 300 is a web page hosted by a server of the computer system 106, and accessed/visited by a client computer of the computer system 106 via the Internet or an intranet. In other embodiments, the user interface 300 is generated entirely by the device or system that stores and runs the PCV application 130.

In the example embodiment of FIG. 3, the use enters three inputs for the model 132 to act upon: the number of days elapsed since a cell culture was introduced to a bioreactor in field 302A, viability (%) in field 302B, and viable cell density (×10⁵ cells per milliliter) in field 302C. The user may enter these inputs via the user input device 124, for example. To infer or predict the packed cell volume, the user selects/activates a control 304 (e.g., via the user input device 124). In response to the user activation of the control 304, the PCV application 130 applies the values entered in fields 302A-302C as inputs to the trained model 132. The model 132 operates on these inputs to infer or predict a packed cell volume value, which the PCV application 130 uses to populate the field 306 for presentation to the user. In embodiments where the model 132 predicts a value or classification that only implicitly indicates packed cell volume (e.g., an indicator of whether a harvesting filter will likely clog), the field 306 may instead depict a value or classification of that sort.

A user may observe the packed cell volume (or other value or classification) shown in the field 306, and take one or more appropriate actions, such as deciding whether the cell culture is suitable for harvesting, manually adjusting various parameters of the harvesting system 112 based on the value and/or classification (e.g., adjust centrifuge timing and/or feed rate), and so on. Conversely, if the parameters in fields 302A-302C represent a purely hypothetical cell culture, the user may simply consider the results shown in field 306, and possibly enter new values, or initiate a real-world experiment based on the results, etc.

In other embodiments, the user interface 300 includes fields for more, fewer, and/or different types of input parameters than those shown in FIG. 3 (e.g., total cell count, cell diameter, viability and/or VCD at each of multiple days, etc.), so long as each of the parameters entered via the user interface 300 is a type of parameter that was used to train the model 132. In some embodiments, however, one or more fields of the user interface 300 relate to parameters that were not used to train the model 132. For example, the memory 126 may store multiple models that are each similar to the model 132, but trained to infer or predict packed cell volume (or a value or classification indicative thereof) for a different molecule type (e.g., a particular antibody). In such embodiments, the user interface 300 may include an additional field in which the user enters the molecule identifier, and the PCV application 130 selects the appropriate model based on the entered molecule identifier.

In some embodiments, the PCV application 130 obtains one, some, or all of the inputs to the model 132 by means other than a user interface. For example, and as discussed in more detail below with reference to FIG. 5, the computing system 106 may be communicatively coupled to one, some, or all of the analytical instrument(s) 104, and automatically retrieve measurements from the analytical instrument(s) 104 to use as inputs to the model 132.

In addition to, or alternative to, presenting an output in a field similar to field 306, the user interface 300 may use any other suitable format or formats for displayed output information, such as graphs, tables, and so on. For example, the computer system 106 may monitor the cell culture in the bioreactor 102 over time, in which case the PCV application 130 may periodically obtain measurements from the analytical instrument(s) 104 and apply those measurements as inputs to the model 132 in order to infer packed cell volume values. In such an embodiment, the user interface 300 may dynamically update a graph or table depicting packed cell volume values over time as those values are determined/obtained.

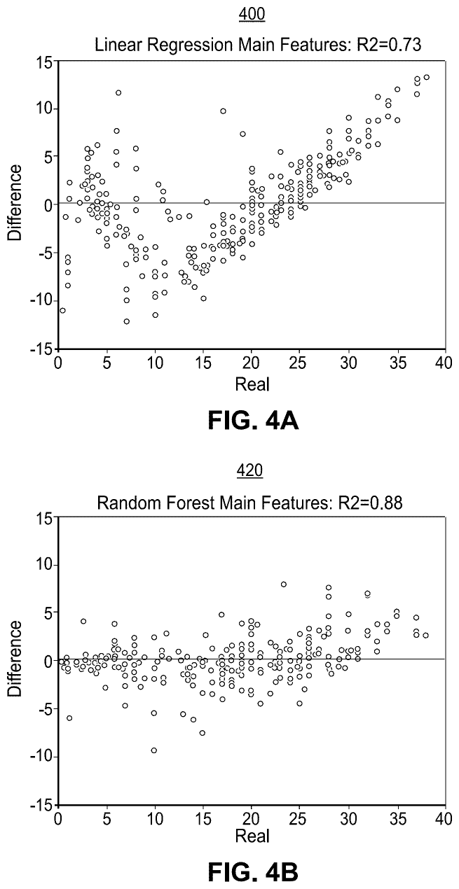

As noted above, the model 132 is a non-linear, machine learning model. While a linear regression model would provide a simpler implementation, the performance of such a model was found to be deficient. Using experiment day (i.e., duration of the cell culture in a bioreactor), total cell count, viable cell density, cell diameter, viability, and osmolality from over 300 datapoints as model inputs (after cleaning the data by removing samples with missing features and/or outliers outside of 2.5 standard deviations), linear regression provided the performance shown in plot 400 of FIG. 4A. In the plot 400, the x-axis represents the real/actual packed cell volume, while the y-axis represents the difference between the inferred packed cell volume and the real/actual packed cell volume (both in %). As seen in FIG. 4A, linear regression resulted in poor performance. The coefficient of regression for the linear regression model was 0.73.

Plot 420 of FIG. 4B shows performance using a non-linear machine learning model (specifically, in this case, a random forest model). As with the plot 400, the x-axis in the plot 420 represents the real/actual packed cell volume, while the y-axis represents the difference between the inferred packed cell volume and the real/actual packed cell volume (both in %). To prepare for random forest machine learning, the historical data was normalized (due to vastly different feature ranges), and then separated into test and training sets at a 4:1 ratio. The random forest regression model was created and trained on this initial data set with the features/predictors/inputs of experiment day, total cell count, viable cell density, cell diameter, viability, and osmolality. However, a feature importance analysis indicated that total cell count, cell diameter, and osmolality were all negligible predictors. The random forest model resulted in the performance shown in plot 420, which represents a coefficient of determination of 0.88. As seen when comparing FIGS. 4A and 4B, the random forest model greatly outperformed the linear regression model. Certain other non-linear models, such as XGBoost, can also substantially outperform a linear regression model.

In some embodiments, in addition to or instead of displaying outputs (e.g., as shown in FIG. 3), the computer system 106 generates control data as an output, to control one or more devices (e.g., components) of the harvesting system 112. FIG. 5 depicts one such embodiment in an example system 500. In FIGS. 1 and 5, the same reference characters refer to functionally similar and/or structurally similar components. As seen in FIG. 5, however, the computer system 106 is communicatively coupled to both the analytical instrument(s) 104 and the harvesting system 112, and the PCV application 130 is replaced with a control application 530.

The control application 530 includes a measurement unit 532, an inference/prediction unit 534, and a controller 536. It is understood that the various units of the control application 530 may be distributed among different software applications, and/or that the functionality of any one such unit may be divided among different software applications. The measurement unit 532 may obtain (e.g., request, or otherwise monitor) the measurements produced by the analytical instrument(s) 104 once, or periodically for any desired number of time intervals (e.g., once per day, once per hour, etc.). In some embodiments, the control application 530 omits the measurement unit 532, and the control application 530 obtains cell culture parameters via user inputs (e.g., via the user interface 300) or by other means.

In some embodiments, the inference/prediction unit 534 infers or predicts packed cell volume values based on the cell culture parameters obtained by the measurement unit 532 (and/or by other means), by applying the cell culture parameters as inputs to the model 132. The controller 536 operates on the inferred/predicted packed cell volume value(s), and possibly also other information (e.g., user-specified or default limits), to generate control data for one or more devices of the harvesting system 112. For example, the computer system 106 may send, to a centrifuge of the harvesting system 112, a command that causes the centrifuge to adjust one or more timing parameters, such as shot frequency (for a bowl shot centrifuge) or solid/liquid split ratio (for a continuous centrifuge). As another example, the computer system 106 may send, to a pump of the harvesting system 112, a command that causes the pump to adjust a feed rate of the contents of the bioreactor 102 to a centrifuge of the harvesting system 112. The controlled device(s) of the harvesting system 112 may include proportional-integral-derivative (PID) controllers, and receive set-points from the computer system 106 (e.g., set points generated by the controller 536) as inputs to the PID controllers, for example. The controller 536 may control the harvesting device(s) before the harvesting process begins and/or during the harvesting process, depending on the embodiment.

In other embodiments, the model 132 directly predicts certain device parameters that are sensitive to packed cell volume, in which case the controller 536 may not need to translate or map packed cell volume values to specific device settings. For example, the model 132 may directly predict a suitable timing parameter value for a centrifuge, in which case the controller 536 simply generates control data that represents that value in a suitable format.

The display device 122 and/or the user input device 124 may be operated by a user to monitor the control operations of the control application 530. For example, the controller 536 may cause the computer system 106 to present the generated device control settings to a user via the display 122, and/or the device(s) of the harvesting system 112 may send settings or readings to the computer system 106 to cause the computer system 106 to present the settings or readings to a user via the display 122.

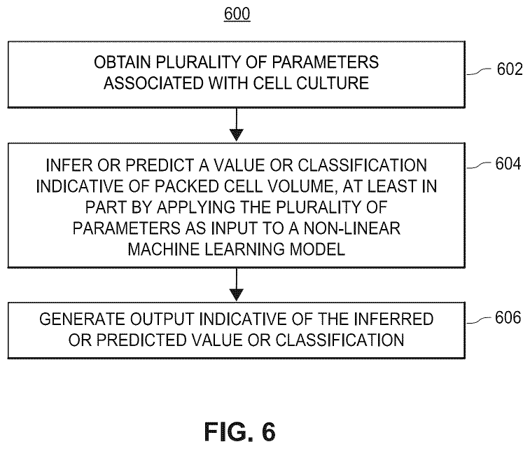

FIG. 6 is a flow diagram of an example method 600 of cell culture assessment. The method 600 may be implemented by a system such as the system 100 of FIG. 1 or the system 500 of FIG. 5 (e.g., by the processing unit 120 executing instructions of the PCV application 130 or instructions of the control application 530). The method 600 may be performed once, or repeated (e.g., in real-time) for one or more time intervals (e.g., each of multiple days) during the cell culture process.

At block 602, a plurality of parameters associated with the cell culture is obtained. The parameters may include analytical instrument measurements (e.g., as measured by the analytical instrument(s) 104 of FIG. 1), for example. In some embodiments, total cell count and/or cell diameter may be specifically excluded from the parameters obtained at block 602. For example, the parameters may consist entirely of the length of time the cell culture was in a bioreactor, viable cell density, and viability. Generally, however, the parameters may include any suitable parameters, such as viable cell density and/or viability on each of multiple days, total cell count, cell diameter, osmolality, molecule type, and so on.

At block 604, a value or classification indicative of packed cell volume is inferred or predicted. Block 604 includes applying the parameters obtained at block 602 as inputs to a non-linear machine learning model, possibly after one or more steps of cleaning, normalizing, and/or otherwise pre-processing the input data. The model (e.g., the model 132) may be a neural network (e.g., a random forest or XGBoost model), for example. In some embodiments, block 604 includes inferring or predicting a packed cell volume value. In other embodiments, block 604 includes inferring or predicting a different value indicative of packed cell volume (e.g., a particular timing parameter for a centrifuge during a harvesting process) or a classification indicative of packed cell volume (e.g., binary indication of whether a filter will likely clog during the harvesting process, or a binary indication of whether the packed cell volume requires modification of one or more centrifuge parameters during the harvesting process, etc.).

At block 606, an output indicative of the inferred or predicted value or classification is generated. For example, block 606 may include generating or populating a user interface (e.g., similar to the user interface 300, or the field 306 thereof) for display to a user (e.g., via the display 122). As another example, block 606 may include generating control data for one or more devices configured to perform at least a portion of the harvesting process (e.g., one or more devices of the harvesting system 112, as discussed above with reference to FIG. 5).

In some embodiments, the method 600 includes one or more additional blocks not shown in FIG. 6. For example, the method 600 may include an additional block, after block 606, in which one or more devices for which control data was generated (at block 606) are controlled in accordance with the generated control data (e.g., by sending the control data to the appropriate device or devices).

Embodiments of the disclosure relate to a non-transitory computer-readable storage medium having computer code thereon for performing various computer-implemented operations. The term “computer-readable storage medium” is used herein to include any medium that is capable of storing or encoding a sequence of instructions or computer codes for performing the operations, methodologies, and techniques described herein. The media and computer code may be those specially designed and constructed for the purposes of the embodiments of the disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as optical disks; and hardware devices that are specially configured to store and execute program code, such as ASICs, programmable logic devices (“PLDs”), and ROM and RAM devices.

Examples of computer code include machine code, such as produced by a compiler, and files containing higher-level code that are executed by a computer using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computer (e.g., a server computer) to a requesting computer (e.g., a client computer or a different server computer) via a transmission channel. Another embodiment of the disclosure may be implemented in hardwired circuitry in place of, or in combination with, machine-executable software instructions.

While the present disclosure has been described and illustrated with reference to specific embodiments thereof, it should be understood by those skilled in the art that various changes may be made and equivalents may be substituted without departing from the true spirit and scope of the present disclosure as defined by the appended claims.

1. A method of cell culture assessment, the method comprising:
obtaining, by one or more processors, a plurality of parameters associated with a cell culture;
inferring or predicting, by the one or more processors, a value or classification indicative of packed cell volume, at least in part by applying the plurality of parameters as inputs to a non-linear machine learning model; and
generating, by the one or more processors, an output indicative of the inferred or predicted value or classification.

2. The method of claim 1, wherein the plurality of parameters includes a plurality of analytical instrument measurements.

3. The method of claim 1, wherein the plurality of parameters includes one or more of:
length of time the cell culture was in a bioreactor;
viable cell density; or
viability.

4. The method of claim 3, wherein the plurality of parameters includes:
length of time the cell culture was in the bioreactor;
viable cell density; and
viability.

5. The method of claim 1, wherein the plurality of parameters excludes:
total cell count; and
cell diameter.

6. The method of claim 1, wherein the non-linear machine learning model comprises a neural network.

7. The method of claim 1, wherein the non-linear machine learning model comprises a random forest model.

8. The method of claim 1, wherein the non-linear machine learning model comprises an XGBoost model.

9. The method of claim 1, wherein inferring or predicting the value or classification includes inferring or predicting a packed cell volume value.

10. The method of claim 1, wherein the method includes inferring or predicting the classification.

11. The method of claim 10, wherein the method includes inferring or predicting whether the packed cell volume exceeds a threshold value.

12. The method of claim 10, wherein the method includes one or both of:
inferring or predicting filter performance during the harvesting process; and
inferring or predicting whether the packed cell volume will require modification of one or more centrifuge parameters during the harvesting process.

13. The method of claim 1, wherein generating the output includes generating or populating a user interface for presentation to a user.

14. The method of claim 1, wherein:
generating the output includes generating control data for one or more devices configured to perform at least a portion of the harvesting process.

15. The method of claim 14, further comprising:
controlling the one or more devices in accordance with the control data.

16. A system comprising:
one or more processors; and
one or more non-transitory, computer-readable media storing instructions that, when executed by the one or more processors, cause the one or more processors to:
obtain a plurality of parameters associated with a cell culture;
infer or predict a value or classification indicative of packed cell volume, at least in part by applying the plurality of parameters as inputs to a non-linear machine learning model; and
generate an output indicative of the inferred or predicted value or classification.

17. The system of claim 16, further comprising:
a plurality of analytical instruments,
wherein the plurality of parameters includes a plurality of measurements obtained by the analytical instruments.

18. The system of claim 16, further comprising:
a bioreactor,
wherein the plurality of parameters includes one or more of (i) length of time the cell culture was in the bioreactor, (ii) viable cell density, or (iii) viability.

19. The system of any ene claim 16, wherein the non-linear machine learning model comprises a neural network.

20. The system claim 16, wherein the non-linear machine learning model comprises a random forest model.

21. The system of claim 16, wherein the non-linear machine learning model comprises an XGBoost model.

22. The system of claim 16, further comprising:
one or more devices configured to perform at least a portion of a harvesting process,
wherein generating the output includes generating control data for the one or more devices, and
wherein the instructions further cause the one or more processors to control the one or more devices in accordance with the control data.
